(19) United States
(12) Patent Application Publication
(10) Pub. No.: US 2007/0160562 A1
(43) Pub. Date: Jul. 12, 2007

(54) Delivery devices for hair-promoting cosmetic agent

(76) Inventor: Michael C. Brinkenhoff, Ventura, CA (US)

Correspondence Address: Craig A. Crandall, APC, 3034 Deer Valley Avenue, Newbury Park, CA, 91320, US

(21) Appl. No.: 11/650,150

(22) Filed: Jan. 5, 2007

Related U.S. Application Data
(60) Provisional application No. 60/757,071, filed on Jan. 6, 2006.

(51) Int. Cl. A61K 31/557; A61K 8/36

(52) U.S. Cl. 424/70.22; 514/573

(57) ABSTRACT

The field of the present invention relates to improved cosmeceuticals capable of stimulating the growth of hair, e.g., natural eyelashes, in a human subject. More specifically, the present invention relates to delivery devices that provide for effective, targeted application of a therapeutically effective amount of the improved cosmeceuticals. Importantly, the targeted application helps to limit and/or eliminate concerns about the cosmeceutical active ingredient effecting unintended areas of the body.

### RELATED PATENT APPLICATIONS

This application claims the benefit of U.S. Provisional Application No. 60/757,071, filed on Jan. 6, 2006, the entire teaching of which is incorporated herein by reference.

### TECHNICAL FIELD

The field of the present invention relates to the use of at least one prostaglandin analog for the manufacture of an improved cosmeceutical which can promote eyelash growth. More specifically, the present invention relates to delivery devices that provide for effective, targeted application of a therapeutically effective amount of the improved cosmeceutical. Importantly, the targeted application helps to limit and/or eliminate concerns about the cosmeceutical active ingredient effecting unintended areas of the body.

### BACKGROUND OF THE INVENTION

A rapidly growing number of American men and women experience some degree of hair loss each year. Hair loss can have a dismaying or even devastating influence on a person's sense of self image and quality of life, leading to psychological and emotional effects that range from decreased self-esteem to anxiety and depression. As a result, men and women spend hundreds of millions of dollars in efforts to grow hair back.

There are many forms of hair loss on the scalp, ranging from alopecia areata to androgenetic alopecia, also known as male or female pattern baldness. Alopecia has many possible causes, including, e.g., genetic disorders, infections, contact with toxic agents, and hormone imbalance. Due to the difficulties in establishing the cause of alopecia, the medical and scientific community has produced an enormous variety of drug and cosmetic treatments. Several hundred U.S. and/or foreign patents have been issued relating to hair loss/alopecia treatment methods and compositions. Unfortunately, no single agent or method of treatment has been found to universally work against all forms of hair loss. Thus, the treatment process is often one of trial and error, leaving the patient searching for the treatment that will produce the desired result for them.

Currently, there are two drugs approved by the Food and Drug Administration to treat alopecia: Rogaine®, also known as minoxidil; and Propecia®, also known as finasteride. Both drugs suffer from modest efficacy and are inconvenient to administer. And, in the case of Propecia®, use has been limited to within male patient populations only. There are also several non FDA-approved topical scalp preparations marketed as hair growth products; see e.g., Folligen, Crinagen, Revivogen, and Tricomin. However, these too have modest efficacy and are generally recommended for use as supplements to Rogaine® and/or Proprecia® treatment.

Other less publicized, yet prevalent hair loss disorders are those involving the eyebrows and/or eyelashes (medarosis). An individual's eyebrows and eyelashes help frame the face. Moreover, the eyebrows and eyelashes protect an individual's eyes by decreasing the amount of light and particles that go into them. As is the case with hair loss on the scalp, when an individual loses his/her beautiful brows or lashes, they tend to feel very self-conscious about their appearance, and their self-esteem and psychological well-being may be negatively impacted.

In addition to the hair loss disorders discussed above, loss of eyebrows and/or eyelashes is associated with a variety of other circumstances and conditions. Such conditions include, e.g., trichotillomania (self-inflicted obsessive plucking or eyebrows and/or eyelashes); medical or surgical treatments such as radiation therapy, chemotherapy, and surgical removal of tumor; autoimmune conditions such as allergic disorders (e.g., allergic response to ibuprofen), thyroid disease, vitiligo, lupus, rheumatoid arthritis, and ulcerative colitis; hormonal changes and stress associated with pregnancy; congenital inability to grow eyebrows and/or eyelashes; and, physical trauma such as thermal, chemical or electrical burns.

Unfortunately, individuals forced to deal with the loss of eyebrows and/or eyelashes have an even more limited number of procedures and treatments available for consideration, as many of treatments described above are either not effective or practical for promoting eyelash growth. Transplantation and reconstructive surgery is one option. However, this is a very specialized procedure that is performed by just a few surgeons. All grafts are single hairs meticulously placed into the lid and itching is a common and troublesome postoperative complication. If the patient gives in to temptation and scratches, there is risk for dislodging the hair grafts and initiating infection.

Many cosmetic and natural products sold over the counter and which rely mainly on coating the existing eyelashes are advertised as having the ability to grow eyelashes, see e.g., MAVALA® Eye-Lite Double Lash, Talika Eyelash Lipocils, Essential Eyelash Formula, Dream Lash™, and NTP Eyelash Enhancer. Again, these products suffer from modest efficacy, can be inconvenient to administer, and may require permanent use to achieve and maintain the desired effect.

In the mid-to-late 90's, prostaglandin analogs (PG analogs), were introduced as intraocular pressure (IOP)-lowering drugs for use in patients with glaucoma and ocular hypertension; see, e.g., U.S. Pat. No. 6,262,105 (the '105 patent) which documents the use of a class of prostaglandins and their derivatives, for lowering intraocular pressure. One such analogue is commonly known as latanoprost, and latanoprost ophthalmic solution (marketed by Pharmacia & Upjohn under the trademark Xalatan®) is an FDA-approved drug. U.S. Pat. No. 5,889,052 (the '052 patent) documents the use of salts and esters of cloprostenol and fluprostenol and various analogues thereof, for the treatment of glaucoma and ocular hypertension. One such fluprostenol analogue is known as travoprost, and travoprost ophthalmic solution (marketed by Alcon Laboratories, Inc, under the trademark Travatan®) is also an FDA-approved drug. U.S. Pat. No. 5,688,819 (the '819 patent) identifies various prostaglandin analogues as potent ocular hypotensives and documents the use thereof as therapeutic agents. One such analogue is known as bimatoprost, and bimatoprost ophthalmic solution (marketed by Allergan, Inc. under the trademark Lumigan®) is also FDA-approved.

An important observation documented with the use of PG analogs in the treatment of glaucoma was the stimulating effects of the drugs on eyebrow and eyelash hair growth and pigmentation; more specifically, observations of increased eyelash growth, thickness, luster and pigmentation; see, e.g., U.S. Pat. No. 6,262,105. In view of this interesting finding, these and other PG analogs have recently been further evaluated for their ability to promote hair growth, including eyelash growth/enhancement; see, e.g., Wolf, et al., 2003 Dermatology Online Journal 9(3): 7, and references cited therein; U.S. patent application Ser. No. 10/275,543 (the '543 application) which broadly discloses methods and compositions for the promotion of hair growth comprising salts and esters of cloprostenol and fluprostenol and various analogues thereof, e.g., travoprost, in various pharmaceutically acceptable carriers suitable for topical administration; and U.S. patent application Ser. No. 10/670,056 (the '056 application) which broadly discloses methods and compositions for thickening, lengthening and darkening eyelashes (in the absence of glaucoma and increased intraocular pressure) comprising latanoprost in an opthalmologically acceptable carrier and medium.

While seemingly excellent candidates to become a drug of choice in promoting eyelash growth, there are several important and serious considerations to address when evaluating the long term safety and overall practicality of using PG analogs solely for the purpose of eyelash enhancement and/or hair growth. For example, additional observations associated with the use of PG analogs to treat glaucoma were darkening of the iris (colored part) and periorbital tissue of the treated eye (the change noticeable usually within several months or years from the start of treatment with the PG analog), as well as increased pigmentation of eyelashes and eyelid skin darkening. And, importantly, these changes to the iris, periorbital tissue, and eyelashes occur only in the eye being treated with the PG analog and may be permanent. As such, if only one eye is being treated, there exists the potential for increased brown pigmentation of the iris, periorbital tissue, and eyelashes in the treated eye only and thus, heterochromia between the eyes; as well as the potential for a disparity between the eyes in length, thickness, and/or number of eyelashes; see, e.g., TRAVATAN® Product Insert.

Other serious safety concerns associated with use of these PG analogs include, e.g., conjunctival hyperemia, ocular pruritus, and various ocular adverse events; Id. As such, it is essential that the medication be delivered such that it will not inadvertently enter into the eye, and allow the intraocular pressure (IOP)-lowering effect of the active ingredient to naturally occur. This is of particular concern when various liquid preparations, e.g., drops, are being contemplated for use. And, to date, liquid preparations have been primarily used in the glaucoma treatment regimens associated with these FDA-approved drugs.

The present inventor believes that in order to utilize the full potential of PG analogs as eyelash enhancers, improved formulations and delivery mechanisms are needed. The present invention provides for such improvements by describing novel preparations comprising PG analogs as an active ingredient. Importantly, these more viscous preparations can be applied with novel, improved delivery devices to provide for a more targeted application, thereby limiting and/or eliminating concerns about the medication effecting unintended areas.

The improvements described herein provide for a more practical and economic use of PG analogs for eyelash enhancement and will be a tremendous benefit to those individuals having to deal with eyebrow/eyelash loss, as well as the many individuals who seek eyebrow/eyelash enhancement for purely cosmetic reasons.

### SUMMARY OF THE INVENTION

One object of the present invention relates to the use of at least one prostaglandin analog as an active ingredient for the manufacture of an improved formulation intended to promote eyelash growth. Importantly, the improved preparation is manufactured such that it can be applied in a targeted manner, thereby limiting and/or eliminating concerns about the active ingredient effecting unintended areas of the body.

Another object of the present invention relates to the development of a cosmeceutical which comprises at least one prostaglandin analog as an active ingredient in a cosmetically or pharmaceutically acceptable excipient, carrier or vehicle. Importantly, the cosmeceutical is manufactured such that it can be applied in a targeted manner, thereby limiting and/or eliminating concerns about the active ingredient effecting unintended areas of the body.

Another object of the present invention relates to providing improved devices for applying the improved preparations of the present invention. The devices are easy to use and provide for specific, targeted delivery of the medication to the intended area.

Yet another object of the present invention relates a method of administering to the skin in the area in which hair growth is desired, an amount effective for achieving said desired effect, of the formulations and/or cosmeceuticals of the present invention.

### BRIEF DESCRIPTION OF THE FIGURES

FIG. 1 is a perspective view of one of the delivery devices contemplated for use in the present invention.

FIG. 2 is a perspective view of one of the delivery devices contemplated for use in the present invention.

FIG. 3 is a perspective view of one of the delivery devices contemplated for use in the present invention.

### DETAILED DESCRIPTION OF THE INVENTION

As those in the art will appreciate, the foregoing detailed description describes certain preferred embodiments of the invention in detail, and is thus only representative and does not depict the actual scope of the invention. Before describing the present invention in detail, it is understood that the invention is not limited to the particular aspects and embodiments described, as these may vary. It is also to be understood that the terminology used herein is for the purpose of describing particular embodiments only, and is not intended to limit the scope of the invention defined by the appended claims.

As used herein, “cosmetics” is defined as articles intended to be rubbed, poured, sprinkled, or sprayed on, introduced into, or otherwise applied to the human body . . . for cleansing, beautifying, promoting attractiveness, or altering the appearance [FD&C Act, sec. 201(i)]. Such products include, e.g., skin moisturizers, perfumes, lipsticks, fingernail polishes, eye and facial makeup preparations, shampoos, permanent waves, hair colors, toothpastes, and deodorants, as well as any material intended for use as a component of a cosmetic product.

As used herein, a “pharmaceutical” is defined as a medicinal drug. The term “cosmeceutical”, as used herein, is a hybrid of the terms cosmetics and pharmaceutical and is understood to define an active ingredient in a cosmetically or pharmaceutically acceptable (suitable for use in a human or other mammal) excipient, carrier or vehicle. The active ingredient is typically one which has been approved for a non-cosmetic use and has been re-formulated for a new consumer use (e.g., uses a lower concentration of the active ingredient than the approved use).

Active ingredients contemplated for use in the improved formulations and/or cosmeceuticals of the present invention include naturally-occurring prostaglandins and/or synthetic prostaglandin analog(s) (PG analogs) described in the art and discussed herein. Prostaglandins are unsaturated carboxylic acids, consisting of a 20 carbon skeleton that also contains a five member ring and are based upon the fatty acid, arachidonic acid. There are a variety of structures: one, two, or three double bonds. On the five member ring there may also be double bonds, a ketone, or alcohol groups. Methods of obtaining and isolating naturally-occurring prostaglandins and/or synthesizing synthetic PG analogs contemplated for use in the present invention are well documented and understood by those skilled in the art.

The present invention relates to the use of at least one prostaglandin analog as an active ingredient for the manufacture of an improved formulation and/or cosmeceutical suitable for topical administration, i.e., applied at a targeted site for exertion of local action, and intended to promote eyelash growth. Accordingly, the formulations and/or cosmeceuticals of the present invention may be presented in the form of aqueous solutions, creams, ointments or oils exhibiting physiologically acceptable osmolarity by addition of pharmacologically acceptable buffers and salts. Importantly, in view of the known effects and various side effects associated with the current use of PG analogs as intraocular pressure (IOP)-lowering drugs, the improved formulations and/or cosmeceuticals of the present invention have viscosity greater than that of simple aqueous solutions so as to decrease variability in delivery the formulations and provide for application in a accurate, specific, targeted manner. As such, the formulations will comprise a pharmaceutically acceptable viscosity building agent. Viscosity building agents contemplated for use in the present invention include, e.g., polyvinyl alcohol, polyvinyl pyrrolidone, and water-soluble cellulosic polymers such as methyl cellulose, hydroxy propyl methylcellulose, hydroxyethyl cellulose, carboxymethylcellulose (CMC), hydroxy propyl ethyl cellulose or other agents known to those skilled in the art. Additional viscosity enhancers contemplated for use include: natural hydrocolloids such as Acacia, tragacanth, alginic acid, carrageenan, locust bean gum, guar gum, gelatin, and hyaluronic acid; and, synthetic hydrocolloids such Carbopol®, PEG, PVP, PVA, pluronics, dextran sulfate. Such agents are typically employed at a concentration between about 0.01% and about 3% by weight. In a preferred embodiment of the present invention, the viscosity building agent was CMC at a concentration of 0.5-1%.

Multi-dose forms of the improved formulations and/or cosmeceuticals of the present invention are also contemplated for use. As such, the formulations may require the addition of preservatives to prevent microbial contamination during use. Suitable preservatives include: benzl alcohol, phenol, cresol, meta-cresol/prophyl cresol, benzalkonium chloride, thimerosal, chlorobutanol, methyl paraben, propyl paraben, phenylethyl alcohol, edetate disodium, sorbic acid, ONAMER M®, phenylmercuric acetate, phenylmercuric nitrate or other agents known to those skilled in the art. Such preservatives are typically employed at a concentration between about 0.001% and about 1.0% by weight.

Due to the limited solubility in water of several prostaglandins and their derivatives, the formulations may require a surfactant or other appropriate co-solvent in the composition. Such surfactants include anionic surfactants chosen, e.g., from optionally unsaturated fatty acid salts having from 12 to 18 carbon atoms, alkali metal salts of salts of organic bases with (C₂-C₁₈) alkylsulfuric acids, alkali metal salts of salts of organic bases with (C₁₂-C₁₈) alkylsulfonic acids, alkali metal salts of salts of organic bases with (C₆-C₁₈) alkylarylsulfonic acids, and ether sulfates; nonionic surfactants, chosen, e.g., from polyalkoxylated surfactants and polyglycerolated surfactants, such as fatty acids, fatty acid amides, fatty alcohols, alkylphenols; esters of fatty acids and polyols, alkanediols, alkyl ethers of alkanediols; and at least one compound chosen from alkyl carbamates of triglycerol, oxyethylenated derivatives of lanolin alcohols, propoxylated derivatives of lanolin alcohols, and lanolin fatty acids; and cationic surfactants, chosen, e.g., from quaternary ammonium derivatives. Such co-solvents include: Polysorbate 20, 60 and 80; Pluronic F-68, F-84 and P-103; Tyloxapol®; Cremophor® EL; sodium dodecyl sulfate; glycerol; PEG 400; propylene glycol; cyclodextrins; or other agents known to those skilled in the art.

The preparations of the present invention may contain additional pharmaceutically acceptable auxiliary substances as required to approximate physiological conditions and as necessary to prepare compositions for convenient administration, such as pH adjusting and buffering agents, and delivery vehicles. For example, tonicity adjustors may be added as needed or convenient. They include, but are not limited to, salts, particularly sodium chloride, potassium chloride, mannitol and glycerin, or any other suitable tonicity adjustor. Various buffers and means for adjusting pH may be used so long as the resulting preparation is pharmaceutically acceptable. Such buffers include acetate buffers, citrate buffers, phosphate buffers and borate buffers. Actual methods for preparing pharmaceutically administrable compounds will be known or apparent to those skilled in the art and are described in detail in, for example, Remington's Pharmaceutical Science, Mack Publishing Co., Easton, Pa. (1985), which is incorporated herein by reference.

Additional ingredients may be added according to the understanding of those familiar with the art in order to vary the texture, consistency, viscosity, and appearance of the formulation. These additional ingredients include emulsifying agents such as non-ionic ethoxylated and nonethoxylated surfactants, fatty alcohols, fatty acids, organic or inorganic bases, preserving agents, wax esters, steroid alcohols, triglyceride esters, phospholipids such as lecithin and cephalin, polyhydric alcohol esters, fatty alcohol ethers, hydrophilic lanolin derivatives, hydrophilic beeswax derivatives, hydrocarbon oils such as palm oil, coconut oil, mineral oil, cocoa butter waxes, silicon oils, pH balancers and cellulose derivatives.

The cosmeceutical may also contain amino acids, polyols, urea, allantoin, sugars and sugar derivatives, water-soluble vitamins, plant extracts (those from Iridacea plants or from soybean) and hydroxy acids (fruit acids or salicylic acid); or lipophilic and chosen from retinol (vitamin A) and its derivatives, especially an ester (retinyl palmitate), tocopherol (vitamin E) and its derivatives (tocopheryl acetate), essential fatty acids, ceramides, essential oils, salicylic acid derivatives, for instance 5-n-octanoyl salicylic acid, hydroxy acid esters, and phospholipids, for instance lecithin, and mixtures thereof.

The improved preparations of the present invention may further comprise at least one additional agent commonly used in cosmetics, chosen, for example, from trace elements, demulcents, sequestrants, perfumes, oils, silicones, thickeners, vitamins, proteins, ceramides, plasticizers, coalescing agents, cohesion agents, alkalinizing agents, acidifying agents, and emollients.

The improved preparations of the present invention may be combined with one or more known agents for the promotion of hair growth. Specifically, the compounds of the present invention may be combined with: i) minoxidil (Pharmacia) and minoxidil-type compounds; ii) finasteride (Merck) and finasteride-type compounds (dihydrotestosterone (DHT) blockers); and iii) copper-peptides or retinoic acid related compounds.

The improved preparations of the present invention may be in the form of a pigmented or unpigmented wax-in-water dispersion, wax-in-oil dispersion, a gelled oil or an aqueous gel mascara, to be applied with an improved device of the present invention. Examples of mascara formulations may be found in U.S. Pat. No. 6,274,131, and references cited therein.

The present invention further relates to providing an improved delivery device for the application of the improved preparations of the present invention. Importantly, the delivery devices provide for easy, accurate and targeted application of the preparations only to the area to be treated.

The delivery device may be a pen-shaped device having a barrel with a reservoir containing the preparation and comprising a slidable push button located on one end of the barrel and an application element at the other end of the barrel. The pen is actuated by depression of the slidable push button which then drives a shaft connected to a piston located within the barrel to drive forward the preparation out of the reservoir and through the application element whereby it is dispensed specifically and directly to the desired area.

The delivery device may comprise a receptacle containing the preparation and an applicator comprising a stem (e.g., a wand) having a application element on one end of the stem and a cap on the other which is intended to cover the receptacle and is attached to the receptacle via the applicator. Manipulation of the applicator is accomplished by simultaneously holding the cap and the receptacle, preferably with both hands, one in each hand. The application element is specifically designed for fine and precise application, preferably for the purpose of creating lines.

The delivery device may be a pen and/or pencil apparatus comprising a first end containing lead or ink for writing, and a second end containing the preparation to be applied and comprising an application element for applying the preparation. The application element may be a tip that is flat, rounded or brush shaped for applying the preparation. The first and second ends of the pen and/or pencil may have caps to cover the ink/lead, or preparation when not in use.

The delivery device may be a pen comprising a pen body having a cavity to contain the preparation; a head portion located at one end of the pen body and having a brush partially protruded out to the exterior thereof, a cap body mounted at one end of the pen body for the capping of the head portion; a application element including a rotational cap, a driving rod, a compression spring, a ratchet wheel, a securing seat, a piston rod and a piston, wherein the rotational cap is mounted at the other end of the pen body and has one end extended to the driving rod within the pen body, and the compression spring and the ratchet wheel are mounted onto the driving rod, and the securing seat is mounted to the pen body with the end of the driving rod, and the bottom face of the securing seat and the top face of the ratchet wheel are provided with mutually engaged teeth, and the end portion of the driving rod has a screw hole and the piston rod is mounted within the screw hole and passes through the securing seat, and the end portion of the piston rod is provided with the piston; and a specified amount of the preparation at the lower section of the head portion of the pen body and the cavity located above the application element.

The delivery device may be a hollow eyeliner brush that, in a manner similar to that of a ball point pen, dispenses with one click a precise quantity of the preparation into the brush-bristles at the tip if the eyeliner brush. This device would provide: (1) precision, because the eyeliner brush itself permits the preparation to be applied in a very precise fashion only to the base of the eyelashes; and (2) quantitative control, thereby reducing the risk of excess active ingredient going into the eye (where it can effect the intraocular pressure, cause redness, and/or change the color of the iris) or onto the skin of the eyelids (where it can cause a darkening of the skin). As such, the device is more economical and user friendly in that the user will feel more confident that they are using the exact amount of the preparation intended to be delivered, with no waste.

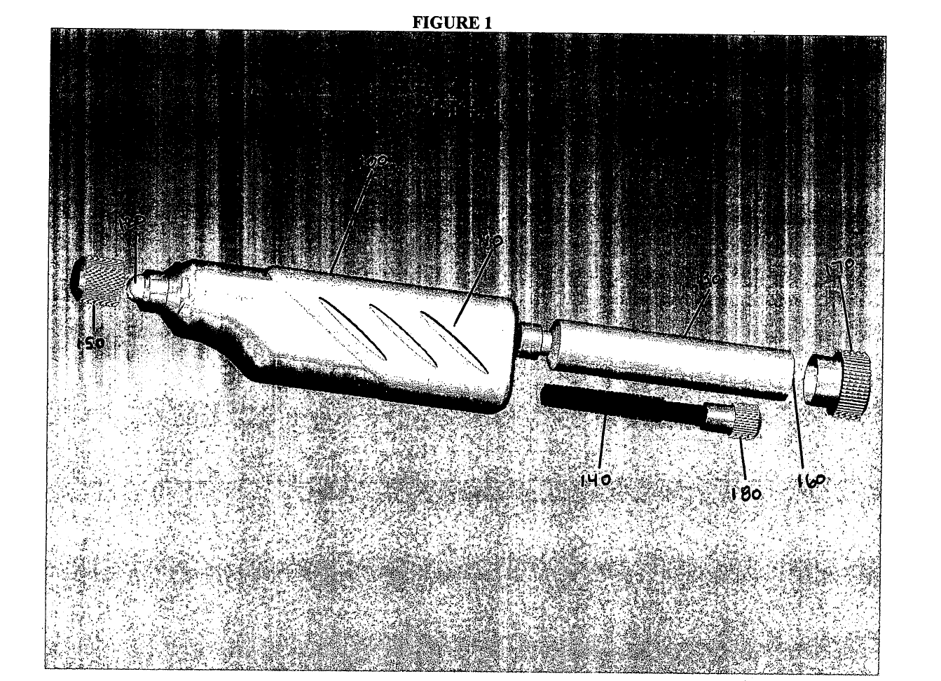

Referring now in more detail to the drawings, FIG. 1 shows a delivery device 100 contemplated for use. The delivery device 100 comprises a receptacle body 110 having a rollerball tip applicator 120 at one end, and capable of receiving a cartridge 130 and a mascara brush 140 at the other end. The rollerball tip applicator 120 has a removable cap 150. The cartridge 130 has an open end 160 which receives the preparation, and has a removable cap 170. The mascara brush 140 has an attached cap 180.

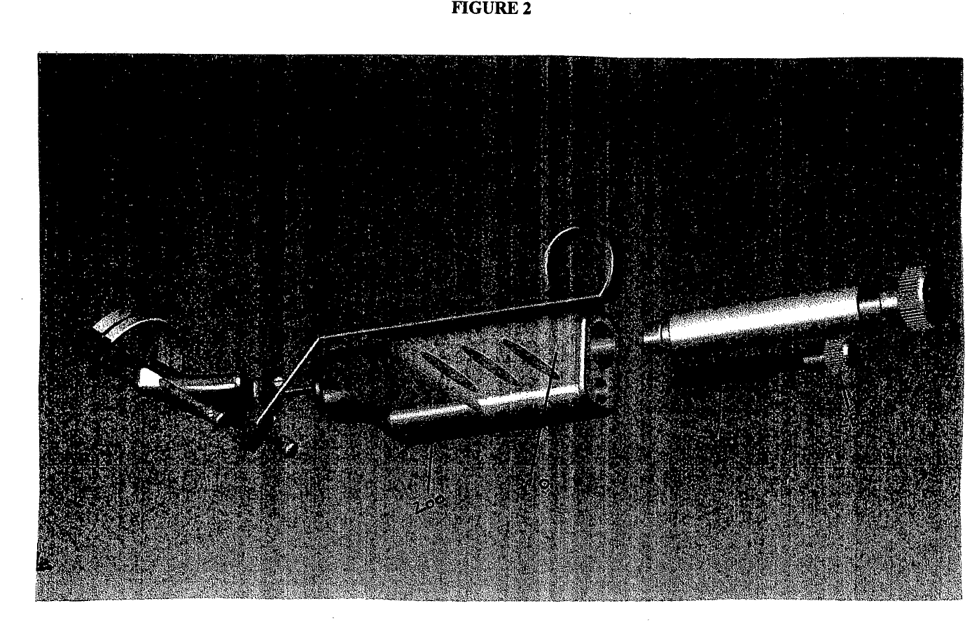

FIG. 2 shows a delivery device 200 contemplated for use. The delivery device 200 comprises a receptacle body 210 having an eyelash curling device 220 at one end, and capable of receiving a cartridge 230 and a mascara brush 240 at the other end. The eyelash curling device 220 has a retractable scissor arm 250 attached thereto which together function to dispense the preparation from the cartridge 230. The cartridge 230 has an open end 260 which receives the preparation, and has a removable cap 270. The mascara brush 240 has an attached cap 280.

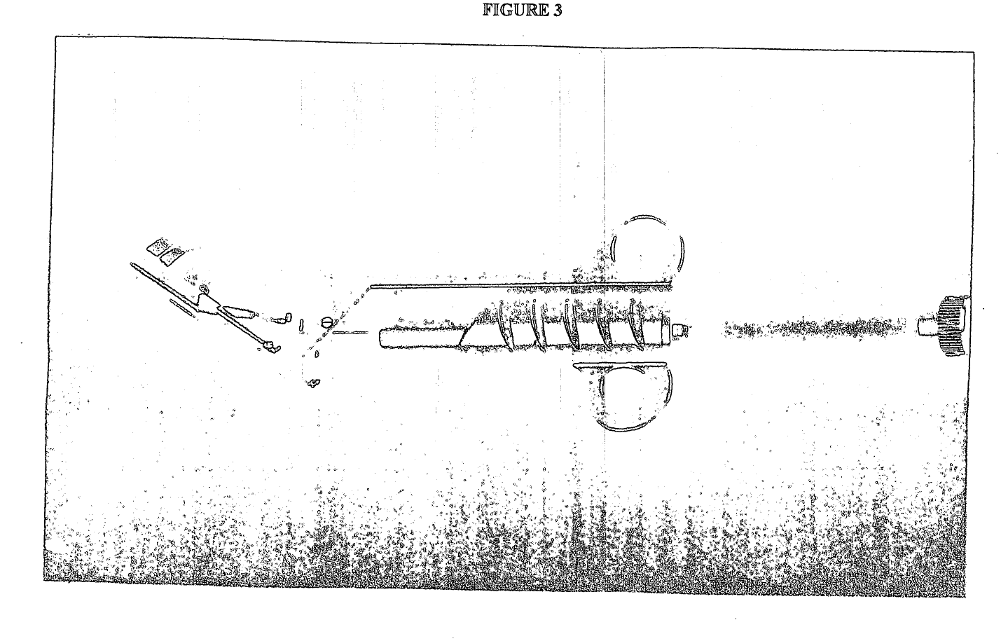

FIG. 3 shows a delivery device 300 contemplated for use. The delivery device 300 comprises a receptacle body 310 having a finger hole attachment 320, and having an eyelash curling device 330 at one end, and capable of receiving a cartridge 340 at the other end. The eyelash curling device 330 has a retractable scissor arm 350 attached thereto which together function to dispense the preparation from the cartridge 340. The cartridge 340 has an open end 360 which receives the preparation, and has a removable cap 370.

The present invention further provides a method of administering to the skin in the area in which hair growth is desired, an amount effective for achieving said desired effect, of the formulations and/or cosmeceuticals of the present invention using a delivery device as described in the preceding paragraphs. Depending on the actual formulation and prostaglandin analogue to be used, various amounts of the drug and different dose regimens may be employed. Typically, the daily amount of prostaglandin for treatment of the eyelid may be about 0.1 ng to about 100 mg per eyelid, more preferably about 1 ng to about 100 μg per eyelid. Effective amounts of the active derivatives will vary depending on the derivative employed, frequency of application and desired result, but will generally range from about 0.0000001% to about 50% by weight of the dermatological preparation. The actual dose of the active ingredients of the present invention depends on the specific compound, and on the condition to be treated. Determination and selection of such appropriate dose is well within the knowledge of the skilled artisan.

### EXAMPLE 1

In this Example, various preparations were prepared and used in studies conducted to demonstrate the efficacy of targeted application of the improved formulations and/or cosmeceuticals of the present invention in promoting eyelash growth. The preparations were prepared in accordance with methods and procedures known and understood by those skilled in the art. The following preparations were prepared: Preparation A—Xalatan® (latanoprost 0.005%), 0.5% carboxymethylcellulose (CMC); Preparation B—Travatan® (travoprost 0.004%), 0.5% carboxymethylcellulose (CMC); and Preparation C—Lumigan® (bimatoprost 0.03%), 0.5% carboxymethylcellulose (CMC).

Protocol. Patients were treated and studied using the following protocol: the preparation was applied to eyelids of the patient once a day for nine weeks. Importantly, the preparations were carefully applied only to the base of the eyelashes using a specialized eyeliner brush. Measurements of the length of each patient's eyelashes were taken prior to treatment (“baseline”), and at time intervals of approximately 3 weeks, 6 weeks, and 9 weeks thereafter. In the studies, ten patients were treated with Preparation A, three patients were treated with Preparation B, and six patients were treated with Preparation C.

Results. Preparation A: with specific reference to the increase in length of the eyelashes above the baseline starting point, the average amount of eyelash growth was not measurable (i.e., no growth beyond baseline starting point) at 3 weeks or at 6 weeks. Average eyelash growth was measured at an average of 0.5 mm (range of 0 mm-1 mm) at 9 weeks. No thickening or darkening of the eyelashes was observed in any of the patients.

Preparation B: with specific reference to the increase in length of the eyelashes above the baseline starting point, the average amount of eyelash growth was measured at 2.5 mm (range: 2 mm-3 mm) at 3 weeks. Average eyelash growth was measured at 7.0 mm (range 4.0 mm-9.0 mm) at 6 weeks. Average eyelash growth was measured at an average of 10.5 mm (range: 8 mm-12 mm) at 9 weeks. All 3 patients experienced a significant and noticeable increase in the length of their eyelashes. In addition, all 3 patients noted both a thickening of their eyelashes as well as a darkening of the lashes, and 2 patients also reported an actual increase in the number of eyelashes along the lid margin.

Preparation C: with specific reference to the increase in length of the eyelashes above the baseline starting point, the average amount of eyelash growth was measured at 2 mm (range: 1 mm-3 mm) at 3 weeks. Average eyelash growth was measured at 6 mm (range from 3 mm-8 mm) at 6 weeks. Average eyelash growth was measured at an average of 10 mm (range of 8 mm-12 mm) at 9 weeks. All 6 patients experienced a significant and noticeable increase in the length of their eyelashes. In addition, all 6 patients noted both a thickening of their eyelashes as well as a darkening of the lashes, and 3 patients also reported an actual increase in the number of eyelashes along the lid margin.

Importantly, one of the patients in the Preparation C treatment group had recently undergone aggressive chemotherapy for a recurrence of metastatic breast cancer. After her chemotherapy, 90% of her previously long eyelashes fell out. The few remaining eyelashes were noted to be sparse, short, light in coloration, and narrow in diameter. After treatment with Preparation C, a remarkable increase in the number, length, and thickness of the eyelashes was noted. These lashes were also noted to be darker in color. In addition, the response to Preparation C was rapid; that is, after 3 weeks, several new eyelashes had begun to sprout, and the remaining eyelashes had grown ˜3 mm. After 6 weeks, many additional new growth sprouts were noted, and there was a significant increase in the length of the existing lashes (8 mm of growth). After 9 weeks, the increased length of the existing lashes was 12 mm. This result suggests the possibility that stimulation of eyelash growth with a prostaglandin analogue, in individuals who have lost their eyelashes due to aggressive chemotherapy, may be an especially effective and helpful treatment, both cosmetically and in terms of helping their self-image.

### EXAMPLE 2

In this Example, a study was performed wherein one patient was treated using Preparation B and Preparation C, alternating daily for a period of nine weeks. Preparation B was applied to the base of the eyelashes once a day on the “even” days of the month, and Preparation C applied to the base of the eyelashes once a day on the “odd” days of the month.

With specific reference to the increase in length of the eyelashes above the baseline starting point, the average amount of eyelash growth was measured at 3.5 mm at 3 weeks. Average eyelash growth was measured at 8.5 mm at 6 weeks. Average eyelash growth was measured at an average of 11.5 mm at 9 weeks. In addition, at 3 weeks the patient noted a significant increase in the number of eyelashes along the lid margin, as well as a darkening and thickening of the new eyelashes. The results of this study suggest that a regime of daily alternating applications of Preparation B and Preparation C to the eyelid margins may be more effective in stimulating new eyelash growth than either preparation by itself.

All of the articles and methods disclosed and claimed herein can be made and executed without undue experimentation in light of the present disclosure. While the articles and methods of this invention have been described in terms of preferred embodiments, it will be apparent to those of skill in the art that variations may be applied to the articles and methods without departing from the spirit and scope of the invention. All such variations and equivalents apparent to those skilled in the art, whether now existing or later developed, are deemed to be within the spirit and scope of the invention as defined by the appended claims. All patents, patent applications, and publications mentioned in the specification are indicative of the levels of those of ordinary skill in the art to which the invention pertains. All patents, patent applications, and publications are herein incorporated by reference in their entirety for all purposes and to the same extent as if each individual publication was specifically and individually indicated to be incorporated by reference in its entirety for any and all purposes. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. An improved cosmeceutical comprising at least one prostaglandin analog in a cosmetically or pharmaceutically acceptable excipient, carrier or vehicle; said cosmeceutical capable of stimulating the growth of natural eyelashes in a human subject.

2. A delivery device for the targeted application to the base of the eyelash of a therapeutically effective amount of a preparation comprising at least one prostaglandin analog in a cosmetically or pharmaceutically acceptable excipient, carrier or vehicle.

3. A method for stimulating the growth of natural eyelashes in a human subject which comprises: applying to the base of the eyelash a therapeutically effective amount of a preparation comprising at least one prostaglandin analog in a cosmetically or pharmaceutically acceptable excipient, carrier or vehicle.
